(19) United States
(12) Patent Application Publication
(10) Pub. No.: US 2022/0178900 A1
(43) Pub. Date: Jun. 9, 2022

(54) BIORELEVANT COMPOSITION

(71) Applicant: BIORELEVANT.COM LTD., London (GB)

(72) Inventors: MATHEW LOUIS STEVEN LEIGH, London (GB); VASCO RAFAEL FERNANDES DOS SANTOS, London (GB); STEVEN LEIGH, London (GB)

(21) Appl. No.: 17/600,679

(22) PCT Filed: Apr. 6, 2020
(86) PCT No.: PCT/GB2020/050904

§ 371 Date: Oct. 1, 2021

(30) Foreign Application Priority Data
Apr. 4, 2019 (GB) 1904757.0

(51) Int. Cl. G01N 33/15; C12Q 1/37; C12Q 1/60; C12Q 1/61

(52) U.S. Cl. CPC G01N 33/15; C12Q 1/61; C12Q 1/60; C12Q 1/37

(57) ABSTRACT

A biorelevant precursor composition suitable, upon dispersing, dilution or suspension in an aqueous medium, for simulating fed-state gastric fluids of mammalian species, wherein said biorelevant precursor composition comprises a substantially solid/sol-id-like concentrate, a viscous gel-like concentrate, or a liquid fat dispersion/concentrate, comprising at least one primary component selected from each of the following groups of primary components comprising: i) Triglyceride and/or diglyceride and/or monoglyceride or any combinations thereof in an amount of from 1-70% by weight; ii) Lecithin and/or lysolecithin in an amount of from 1-45% by weight; iii) Carbohydrate in an amount of from 15-70% by weight; and iv) Water or other aqueous medium in an amount of from 1-70% by weight; wherein the weight ratio of total fats (one or more primary components from each of groups i) and ii) combined): total carbohydrates (one or more primary components from group iii) combined) is between 20:1 to 1:20; and the weight ratio of glyceride:lecithin and/or lysolecithin is between 45:1 and 1:45; and in addition at least one additional component selected from at least one of the following: (i) fatty acids (between 0.01-15% by weight); (ii) bile acid/salt (between 0.01-3% by weight); (iii) enzymes (between 0.01-2% by weight); (iv) cholesterol, sterols (between 0.01-5% by weight); (v) buffer agents (between 0.01-4% by weight); (vi) osmotic agents (between 0.01-10% by weight); 52 (vii) proteins (collagen, protein hydrolysates, amino acids) (between 0.01-30% by weight); (viii) mucin (between 0.1-5% by weight); (ix) viscosity modifier (between 0.1-5% by weight); and (x) preservatives, stabilizers (between 0.01-3% by weight), such as a) anti-oxidants, b) chelating agents, c) buffers (inorganic or organic), and d) antimicrobials; all percentages being by dry weight. A method of producing these compositions is also provided.

### FIELD OF THE INVENTION

This invention is in the field of drug solubility, dissolution and biorelevant testing. The invention describes concentrates and dissolution compositions for preparing in vitro biorelevant test media. The in vitro media comprise biologically relevant dietary components and share the physical and chemical parameters of gastric fluids, particularly human gastric fluids, after food consumption. The synthetic in vitro media simulate fed state gastric fluids and can be used for biorelevant solubility, dissolution and stability profiling of drugs and dosage forms, in vitro comparative dissolution testing, in vitro-in vivo correlations, and in vitro studies in simulated gastric fluids for food effects on drugs.

### BACKGROUND TO THE INVENTION

The major dietary components in food comprising fats (including oils, for example glycerides and lipids, for example phospholipids), carbohydrates, and proteins, provide nutrition and support biological functions.

In the stomach, where ingested food is stored and digested, lipophilic components, for example drugs, may be solubilised in (partially) digested dietary fats along with food components, gastric enzymes and secretions, before the chyme is transferred to the upper small intestine. The upper small intestine comprises duodenum, ileum and jejunum where further digestion and absorption takes place, catalysed by enzymes responding to food. In addition, wetting and solubilisation of lipophilic components, including drugs, are assisted by lipolysis products in association with biological surfactants such as bile salts and lecithin. Residence in the stomach before the chyme, containing partially digested food, enters the upper small intestine, may be about 30 minutes to more than 6 hours, depending on the food components and drug/dosage form.

Reference herein to the gastrointestinal (GI) tract includes the stomach and upper small intestine region. This invention concerns simulated fed state gastric fluids induced by, for example, a high-fat (for example containing 55 g to 65 g of fat) or low-fat meal (for example containing 11 g to 14 g of fat) as recommended by the FDA to assess the effects of food on drugs (Draft Guidance for Industry 2019). The invention provides fed state gastric media for in vitro solubility and dissolution testing and can help with understanding how a drug is released from the dosage form in the stomach after a meal. The biorelevant simulated fed state gastric fluids of this invention for use in in vitro dissolution tests are referred to as FEDGAS. These tests may help forecast food effects on drug absorption which may be positive, negative or remain the same. Solubility and dissolution/dissolution rate are key parameters for oral drug absorption and subsequent bioavailability. Solubility and dissolution testing in FEDGAS fed state dissolution media compared to those in the fasted state simulated gastric fluids can help predict if oral absorption could be affected by food.

The tests can also be used to de-risk bioequivalence studies by making sure the dissolution profiles of a generic test product match the reference listed (innovator) drug.

The physicochemical properties of the stomach environment, for example pH, buffer capacity, osmolality, ionic strength and motility, can change significantly in response to food intake. Pharmacokinetic parameters such as Cmax, Tmax, and AUC can be correlated with solubility and dissolution profiles in the fed state simulated gastric fluids (FEDGAS). In combination with biorelevant in vitro dissolution testing in simulated fed and fasted states intestinal fluids and coupled with physiologically based pharmacokinetic modelling, drug bioavailability can be forecasted. Modified and extended release dosage forms with pH-dependent coatings in the GI environment, for example between pH 4.5-6.5, may have different disintegration times and drug release patterns in vivo due to the fed state gastric fluids typically spanning a wide physiological pH range. This behaviour can be reflected in in vitro dissolution media (pH 3, pH 4.5 and pH 6.0) testing using fed state simulated gastric fluids (FEDGAS). The invention is also suitable for in vitro dissolution testing of other modified released formulations including but not limited to, for example, diffusion systems, dissolution systems, osmotic systems, ion-exchange resin, floating systems, bio-adhesive systems and matrix systems.

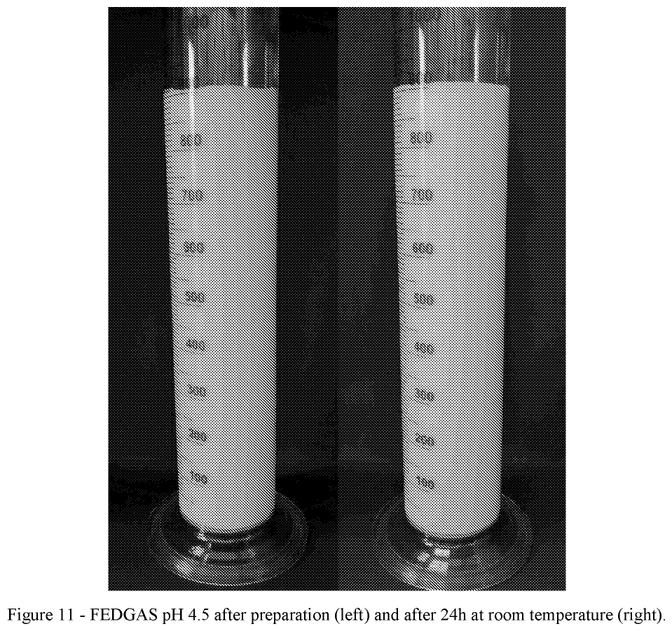

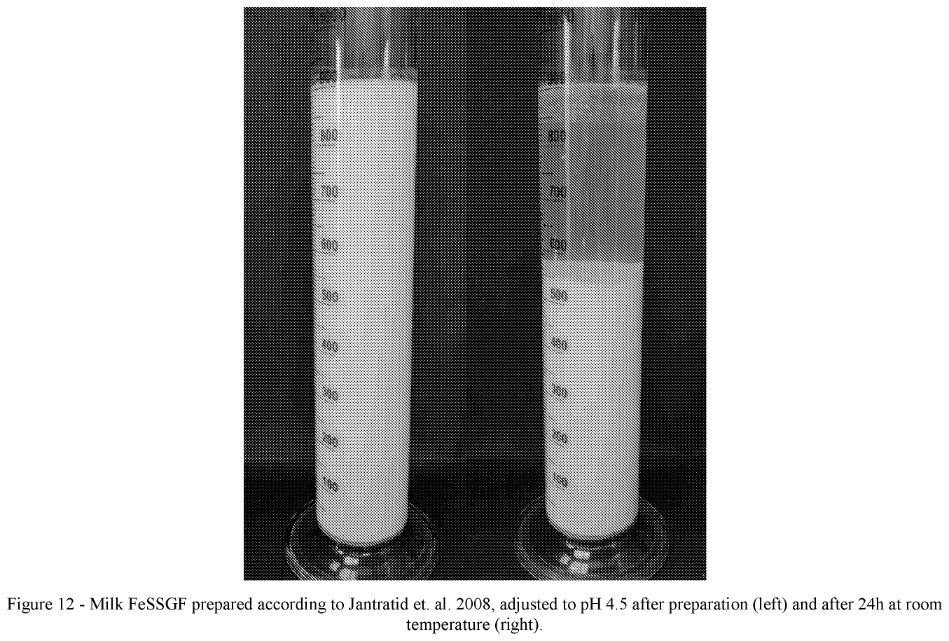

The physicochemical stability of FEDGAS does not change in in vitro dissolution testing for more than 24 hours at 37° C. Thus the extended release dosage forms may be tested in FEDGAS dissolution media for more than 24 hours at 25° C. and 37° C. which further supports the advantages and industrial applicability of the invention in comparison to the prior art dissolution tests typically limited to 8 hours (see FIG. 11 and FIG. 12). The test media are particularly useful for equilibrium solubility testing where equilibrium may take longer than 24 hours.

Accordingly, this invention focuses on biorelevant in vitro gastric media with reproducible criteria (for example, particle size and filterability) more closely mimicking and simulating the physicochemical properties of human gastric fluids after food intake.

The composition and physical properties of the fed state GI fluids depend on the type of food (cf the Food & Drug Administration (FDA) recommended standard high-fat meal and low-fat meal as described in the 2019 draft guidance on Assessing the Effects of Food on Drugs in INDs and NDAs) and the length of time in the stomach. In the fasted state without food, gastric fluid typically presents with a low pH around pH 1-3. After a meal, the pH rises to around pH 6, before returning to base levels between pH 1-3 in up to about 6 hours. Changes in gastric pH mostly affect weak acids and weak bases with the increased values in the fed state enhancing dissolution of acids and reducing the dissolution of bases at pH 6, but the converse may be true at pH 3.

Compendial media simulating gastric fluid (for example in USP and EP) do not contain physiological/biological components (such as fats) and are generally not suitable for biorelevant solubility and dissolution testing of water insoluble drugs.

In one aspect, the in vitro fed state dissolution media (FEDGAS) derived from the precursor concentrates simulate in vivo gastric fluids after eating, for example, an FDA high-fat meal. This high-fat meal is a standard recommended high-fat meal eaten for carrying out in vivo trials of food effects with drugs and bioequivalence for in vivo food effect human studies.

In another aspect, said fed state gastric media (FEDGAS) for in vitro dissolution testing comprise the amount of fat present in a meal. The in vitro dissolution media prepared from the precursor concentrates can provide the amount of fat similar to the amount of fat in the meal taken (between 1 g and 200 g).

It is to be appreciated that the in vitro fed state gastric test media described in this invention have practical uses and industrial applicability not limited to solubility and dissolution testing. For example, the in vitro biorelevant media can be used for testing compatibility of gastric implants, devices and bands; and assessing stability of probiotics and vitamin products to ascertain if they are labile and can survive in the stomach with food.

This invention is concerned with the development and provision of in vitro fed state simulated gastric fluids (FEDGAS) based on, for example, the FDA high-fat and low-fat meals and other meal variants (for example Klein et al., “Media to simulate the postprandial stomach I. Matching the physicochemical characteristics of standard breakfasts.” Journal of pharmacy and pharmacology 56(5) (2004): 605-610) that are recommended for in vivo human studies to assess the effect of food on drugs, for example (i) high-fat high-calorie, (ii) medium-fat medium-calorie, (iii) low-fat low-calorie and (iv) low or high calorie meals with similar fat amount.

### PRIOR ART

F. Baxevanis et al., European Journal of Pharmaceutics and Biopharmaceutics, Vol. 107, 2016, 234-248, describe physicochemical factors affecting drug solubility and dissolution in various simulated gastric fluids after food. Simulated fed state gastric fluids for dissolution tests and drug analysis techniques are comprehensively reviewed. Fed conditions can have significant effects on in vitro drug dissolution and subsequent absorption profiles. It was highlighted that drug analyses in prior art fed state gastric media can be challenging since most analytical protocols employed are time consuming and labour intensive. Because of the physical nature and chemical compositions of prior art dissolution media, filterability and appropriate buffer (avoiding precipitation and enabling 100% drug recovery) are pressing issues that need practical consideration in in vitro tests. Clearly, there is an unmet need for more user-friendly biorelevant dissolution media that can better simulate the gastric fluids after meals, avoiding the problems identified of, for example, compatibility, precipitation, filterability and drug recovery across physiological gastric fed state pH (for example, between pH 1.5 to 7.5) in prior art in vitro dissolution testing.

In Jantratid et al., Pharmaceutical Research, Vol. 25, No. 7, 2008, 1663-1676, a snapshot fed state simulated medium (FeSSGF) is proposed for dissolution tests, comprising 50% acetate buffer and 50% UHT milk (max 3.5% fat in the full fat UHT milk) giving a fat amount of 1.75% at pH 5.0 in FeSSGF. Three “snapshot” media (Early, Middle, Late) compositions to simulate fed state simulated gastric fluid corresponding to pH 6.4, 5 and 3 are shown in Table 2 of Jantratid et al. FeSSGF is the acronym given to identify the mid stage, middle fed state simulated gastric fluid at pH 5, between 75 minutes to 165 minutes after eating. However, this composition referred to as Middle (FeSSGF) in the aforementioned Table 2 essentially contains milk/buffer and does not have the fat amount of an FDA high-fat meal and is affected by variations in the amount and quality of fat in the milk. Furthermore, the physical stability is unsatisfactory (see photograph 4 of Jantratid et al.). Table 4 in Jantratid et al. shows a dissolution medium to simulate the fed state upper small intestine, whilst the present invention describes media simulating the fed state stomach. For the avoidance of doubt, FEDGAS simulating the fed state stomach fluids in this invention, and FeSSIF (Table 4 of Jantratid et al.) to simulate the fed state upper small intestinal fluids, are not similar in vitro dissolution compositions.

US2016/0299113A1 describes solid compositions for preparing biorelevant dissolution media simulating fasted and fed states intestinal fluids for dissolution testing. The disclosure teaches bile salt to phospholipid mole ratios between 1:1 and 20:1 and so the products contain bile salt amounts which are outside the scope of the present invention.

In vitro dissolution testing in prior art simulated gastric fluids include EnsurePlus® (a commercially available “protein milk shake” drink) containing fat (4.6% w/v total fat), proteins and carbohydrates simulating the fed state gastric fluid. This media is not suitable for in vitro dissolution testing because like full fat milk it is not stable across the physiological pH range of the fed stomach and filtration is extremely difficult using a 0.45 micrometer filter, for example using a GD/X Glass Micro Fiber (GMF) Syringe Filter.

Similarly, FeSSGF mimicking fed state gastric fluids is also unsuitable as an in vitro dissolution medium (discussed previously) because it is difficult to filter and physically unstable across the physiological gastric pH range. Furthermore, FeSSGF does not contain the amount of fat found in the FDA high-fat meal.

A further example of prior art FeSSGF medium for dissolution studies is “fed state simulated gastric emulsion” (FeSSGEm), a mixture of an acetate buffer and Lipofundin MCT® made up in a ratio of 82.5:17.5. The medium comprises triglycerides (1.75%), egg lecithin (about 0.21%) and glycerine (0.44%). Significantly, the composition does not contain the amount of fat and carbohydrates in particular the recommended FDA high-fat recommended meal and could be unstable across the pH range of the fed stomach (Klein, In vitro lipolysis assay as a prognostic tool for the development of lipid based drug delivery systems: Martin Luther Universitat Halle-Wittenberg; 2013).

### SUMMARY

The physicochemical properties and performance of various fed state gastric dissolution media are summarised and compared in Table 1 below. It will be appreciated that Table 1 includes the fed state gastric media FEDGAS of the present invention, and clearly shows that the said FEDGAS ticks all boxes (+) and thus meets all of the identified parameters lacking (−) in the known prior art media.

TABLE 1
Comparison of the physicochemical properties and performance of fed
state in vitro dissolution media for example FeSSGF and the proposed in vitro fed
state gastric media (FEDGAS) of this invention.

| | | | | Suitability for | |
|---|---|---|---|---|---|
| | | | | filtration and | |
| | | | | reproducible | |
| | | | | biorelevant | |
| | | Fat amount | Media | dissolution | |
| | | in FDA | physical, | testing in USP | Preparation |
| | Media | high-fat | chemical | Dissolution | time |
| | pH | meal | stability | Apparatus 2 | (<5 min) |
| Proposed fed state | 3 | + | + | + | + |
| gastric media of the | 4.5 | + | + | + | + |
| invention | 5 | + | + | + | + |
| (FEDGAS) | 6 | + | + | + | + |
| Homogenised FDA | 3 | + | − | − | − |
| meal | 5 | + | − | − | − |
| | 6 | + | − | − | − |
| Full fat milk | 3 | − | − | − | − |
| | 4.5 | − | − | − | − |
| | 5 | − | − | − | − |
| | 6 | − | − | − | − |
| FeSSGF (Full fat | 3 | − | − | − | − |
| milk/Buffer) | 5 | − | − | − | − |
| | 6 | − | − | − | − |
| Ensure plus ® | 3 | − | − | − | − |
| | 5 | − | − | − | − |
| | 6 | − | − | − | − |
| FeSSGEm | 3 | − | − | − | − |
| (Lipofundin | 5 | − | + | − | − |
| MCT ®) | 6 | − | + | − | − |

+ Suitable to use as test media as described.
− Not suitable to use as test media as described.

As shown in Table 1, liquid enteral and parenteral products along with the actual homogenised standard FDA meal have been investigated as in vitro dissolution media simulating gastric fed state conditions. Notwithstanding, problems of extraction and the time taken for analysis of the drugs require more user friendly simulated fed state gastric fluid, compatible with standard USP dissolution equipment and recommended method. Little information is available to suggest that prior art dissolution media are fit for purpose in drug dissolution testing lasting up to more than 4 hours at 37° C., and also compatible across the physiological gastric fed state pH range between pH 7.5 to pH 1.5, spanning “early” through to “middle” to “late” stages of digestion in the stomach. Tests at the three typical pHs across the pH range 7.5 to 1.5 are necessary so that solubility and dissolution profiles can be monitored whilst the drug is in contact with food in the fed stomach.

It is an object of this invention to provide in vitro dissolution media containing dietary components comprising principally fats and carbohydrates, which capture and replicate the physicochemical properties of gastric fluids after food. Furthermore, the dissolution compositions typically contain the amounts of fat and carbohydrate present in the FDA recommended high-fat, high-calorie standard meal taken when in vivo BA/BE (Bioavailability/Bioequivalence) studies and food effects on drugs in humans are carried out. There is as yet an unmet need for in vitro dissolution media comprising biorelevant components replicating or capable of replicating the physical and chemical properties of fed state human gastric fluids after ingestion of meals with variable fat content ranging from 1 g to 200 g.

This invention discloses in vitro biorelevant dissolution media dedicated to testing for food effects in the stomach after meals, including but not limited to solubility, stability, dissolution profiling of new chemical entities as well as generic drugs, dissolution comparisons of dosage forms to support BA/BE studies, compatibility across the pH range in fed stomach contents for probiotics, nutritional supplements, vitamins, gastric implants, device-performance and safety tests. In addition to labour-saving benefits, consistency and reproducibility, the fed state gastric media are compatible with filtration after incubation, thereby allowing HPLC analysis of the drug or breakdown products in the filtrate, highlighted in the aforementioned prior art (Baxevanis, et al.).

It is submitted that compared to the media described in the prior art (for example FeSSGF), the biorelevant fed state gastric media (FEDGAS) described herein are better suited for in vitro dissolution testing and in vitro-in vivo correlations, given FEDGAS simulate in vivo gastric fluids after consumption of, for example, the FDA standard high-fat meal.

### BRIEF DESCRIPTION OF THE INVENTION

The term “biorelevant concentrate/composition” is also referred to as the “biorelevant precursor composition” or “biorelevant precursor concentrate” herein.

The present inventors have found that the in vitro gastric dissolution media comprising dispersed fats along with carbohydrates simulate the properties of gastric fluids after consumption of for example a high fat meal. Fats include triglycerides, diglycerides, monoglycerides, lecithin and/or lysolecithin.

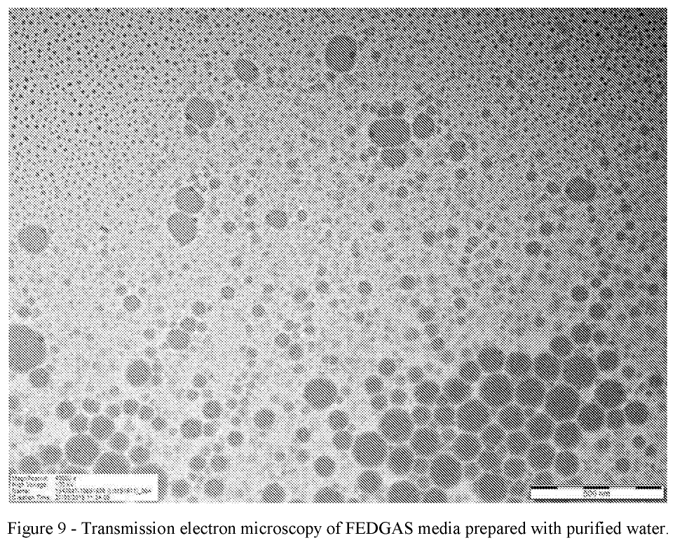

This invention provides substantially solid/solid-like concentrates, substantially clear to opaque viscous, gel-like compositions (concentrates) and fat dispersion/liquid concentrates containing high levels of fats and carbohydrates, wherein the combinations of high energy input and the components comprising chiefly triglycerides, lecithin and/or lysolecithin and carbohydrates produce readily water dispersible fat dispersions (concentrates). Dilution of these readily water dispersible concentrates with aqueous media comprising, for example, buffers and osmotic components yield fed state dissolution media and fat aggregates below 500 nm Z-average diameter (FIG. 9). Unexpectedly, there is afforded exemplary physicochemical stability in in vitro dissolution testing, for at least 24 hours after preparation of the media, and long-term stability of the concentrates for up to at least 12 months at room temperature.

Appropriate buffers are selected from but not limited to acetate, phosphate and citrate buffers to span the fed state physiological gastric pH range from 1.5 to 7.5. Particularly useful pH parameters are at pH 3.0, pH 4.5, pH 5.0 and pH 6.0 reflecting the pH of human gastric fluids after intake of a high-fat or low-fat meal at different residence times with food in the fed stomach. The in vitro dissolution media prepared from the concentrate compositions also avoid the analytical problems such as filtration and pH incompatibility across the fed state stomach pH between pH 1.5 to pH 7.5, identified in the prior art and set out in Table 1.

The process involves high energy input, controlled evaporation and/or careful addition of a target amount of water.

Processing steps comprise:
(i) high energy input, for example high pressure emulsification and the like, sufficient to produce finely divided and substantially uniform fat particles/aggregates below 1000 nm average diameter, typically below about 500 nm; and
(ii) controlled evaporation after addition of water containing at least one carbohydrate such as a sugar or directly controlling the addition of water containing at least one carbohydrate, such as a sugar, wherein the water content of the resulting target composition is closely controlled between 1.0% and 70.0% by weight, typically between 1.0% and 10.0% for a solid/solid-like concentrate, typically between 10% and 25% for a viscous gel-like concentrate, typically between 25% and 70% for a fat dispersion/liquid concentrate.

The process and process steps detailed herein constitute another aspect of the invention.

The substantially solid/solid-like concentrates, viscous gel-like concentrates compositions and liquid fat dispersion concentrates obtained are concentrates as well as precursors for preparing the fed state biorelevant dissolution media as described.

The test media can be prepared by dispersing, diluting or suspending the readily water dispersible precursor concentrates with aqueous media using simple mixing (for example a magnetic stirrer) and without any high energy input. The resulting test media are stable uniformly dispersed fat dispersions readily filterable through a 0.45 micrometer filter.

The media comprising uniformly finely dispersed fat particles are suitable for dissolution testing between pH 1.5 and about pH 7.5, thereby providing pH compatibility across this fed state gastric physiological pH range which has hampered wider use of prior art fed state stomach dissolution media. In addition, pH incompatibility, filterability and reproducibility constraints associated with, for example, milk and enteral surrogates have frustrated in vitro dissolution and testing for food effects using conventional dissolution equipment including but not limited to, for example, USP Dissolution Apparatus 2.

For solubility tests, volumes between 0.5 mL to 20 mL, more typically 5 mL to 10 mL, may be used to assess kinetic and equilibrium solubility determined, for example, by the shake flask method.

For standard USP Dissolution Apparatus 1 and 2 vessel volumes between 250 mL to 1 L, preferably 750 mL to 900 mL, may be used. For in vitro dissolution testing mini-dissolution vessels with lower volumes, typically from 50 to 200 mL, may be used.

In tests where a flow through study using for example a USP Dissolution Apparatus 4 is necessary, the amount of dissolution media can be increased to several litres if an open loop test is required.

The development of the suitable biorelevant in vitro fed state gastric dissolution media which can be used with a simple and robust analytical method, such as HPLC, requiring separation of undissolved drug by filtration, introduce a useful means of testing food effects on drugs and drug products. Several fed state media such as milk, nutrient drinks or Fed State Simulated Gastric Fluid (FeSSGF) at pH 5 have been trialled in the prior art to simulate the human postprandial conditions (Table iTable 1). However, until now, none have managed to achieve satisfactory representation of the actual fed state gastric fluids after FDA meal and are impractical for routine lab use. The proposed biorelevant dissolution media (FEDGAS) of this invention more precisely mimic human gastric fluids after a high-fat meal corresponding to pH 6.0 at about 30 minutes and returning to base levels of pH 3.0 about 6 hours after eating. However, due to the varying composition of meals and the inter- and intra-variability in response to the fed state human gastric physiology the pH may span a wider range, for example between pH 7.5 to pH 1.5. In one aspect, the invention provides a biorelevant composition in a concentrate suitable for preparing the in vitro fed state dissolution medium, upon dispersion or dilution in an aqueous medium, for simulating fed state gastric fluids of mammalian species.

In one embodiment of the present invention, the aqueous medium for dispersing or diluting the biorelevant concentrate composition for preparing the in vitro fed state dissolution media may comprise from ×3 to ×60 dilutable buffer concentrate.

In one embodiment, the biorelevant precursor composition of this invention is a substantially solid/solid-like concentrate, a viscous gel-like concentrate, or a liquid fat dispersion/concentrate, comprising at least one primary component selected from each of the following groups:
i) Triglyceride and/or diglyceride and/or monoglyceride or any combinations thereof in an amount of from 1-70% by weight;
ii) Lecithin and/or lysolecithin in an amount of from 1-45% by weight;
iii) Carbohydrate in an amount of from 15-70% by weight; and
iv) Water or other aqueous medium in an amount of from 1-70% by weight;
   wherein the weight ratio of total fats (one or more primary components from each of groups i) and ii) combined):total carbohydrates (one or more primary components from group iii) combined) is between 20:1 to 1:20; and
   the weight ratio of glyceride:lecithin and/or lysolecithin is between 45:1 and 1:45; and
   in addition, at least one additional component selected from at least one member of the group comprising or consisting of:
(i) fatty acids (between 0.01-15% by weight);
(ii) bile acid/salt (between 0.01-3% by weight);
(iii) enzymes (between 0.01-2% by weight);
(iv) cholesterol, sterols (between 0.01-5% by weight);
(v) buffer agents (between 0.01-4% by weight);
(vi) osmotic agents (between 0.01-8% by weight);
(vii) proteins (collagen, protein hydrolysates, amino acids) (between 0.01-30% by weight);
(viii) mucin (between 0.1-5% by weight);
(ix) viscosity modifier (between 0.1-5% by weight); and
(x) preservatives, stabilizers (between 0.01-3% by weight), such as
   a) anti-oxidants,
   b) chelating agents,
   c) buffers (inorganic or organic), and
   d) antimicrobials.

Unless indicated to the contrary, all percentages by weight (referred to above and below) are by dry weight.

In one embodiment of the present invention, the aqueous medium for dispersing or diluting the biorelevant concentrate composition for preparing the in vitro fed state dissolution medium may comprise:
(i) a buffer solution to reach the required pH, buffer capacity and osmolarity of the biorelevant dissolution media without further dilution; or
(ii) a sufficient weighed/measured amount of the biorelevant concentrate, a sufficient weighed/measured amount of the buffer concentrate, and purified water to reach the required pH, buffer capacity and osmolarity, in any order of incorporation and/or dilution.

In another embodiment of the invention, for example mucin, enzymes (for example, pepsin and/or pancreatin) and/or proteins and/or amino acids, reflecting the contents of the high-fat and low-fat meals may be separately added to the in vitro biorelevant fed state gastric dissolution media either during or after preparing the said fed state dissolution media. Alternatively, the components may also be added to a buffer concentrate or added to the diluted buffer solution.

### Size Reduction and Controlled Evaporation or Titration

In another aspect, the invention provides a process of making a precursor concentrate which process comprises processing dietary components and uniformly dispersing and/or homogenising and/or controlling water content of between 1.0% and 70.0% by weight through evaporation, for example vacuum evaporation or thin film evaporation, dialysis, microwave, and/or addition or titration, obtaining a substantially solid/solid-like composition; viscous gel-like composition and liquid fat dispersion/concentrate.

In one embodiment, the substantially solid/solid-like concentrates typically contain between 1% and 10% by weight of water or aqueous medium.

In one embodiment, the viscous gel-like concentrates typically contain between 10% and 25% by weight of water or aqueous medium.

In one embodiment, the liquid fat dispersion concentrates typically contain between 25% and 70% by weight of water or aqueous medium.

Method of Making Biorelevant in vitro Dissolution Media from Concentrates

As explained herein, the concentrate or biorelevant precursor composition, based on the fat amount of the recommended FDA standard high-fat or low-fat meal and alternative meals, can be converted into a biorelevant in vitro fed state gastric dissolution medium by adding an appropriate aqueous medium or diluent. Accordingly, in one aspect, the invention further provides a method of making a synthetic biorelevant in vitro fed state dissolution medium based on the amount of fat in a high fat to low fat meals comprising adding an aqueous medium, buffered to from pH 1.5 to pH 7.5 or adding from ×3 times to ×60 times buffer concentrate to the biorelevant precursor concentrate composition of the invention. The invention also provides a synthetic in vitro biorelevant fed state dissolution medium comprising the biorelevant precursor concentrate composition of the invention and an aqueous medium. The invention also provides a synthetic in vitro biorelevant fed state dissolution medium obtainable by adding an aqueous medium to the biorelevant precursor concentrate of the invention.

Aqueous medium comprises, for example, purified water and may also comprise aqueous solutions of buffers, from ×3 to ×60 buffer concentrates, osmotic components, ethanol, stabilisers, enzymes. The buffers preferably comprise one or more inorganic or organic buffer agents selected from the list herein.

Typically, when the biorelevant concentrate/composition is a liquid fat dispersion concentrate or substantially clear to opaque viscous gel-like composition, or substantially solid/solid-like concentrate, said dispersion or dilution in an aqueous medium involves contacting the precursor composition with at least the same volume of aqueous medium. Preferably, said precursor composition is diluted with from at least two times the volume of aqueous medium to at least ten times the volume of aqueous medium. For media with low fat content an even higher dilution (up to ×100 times) is within the scope of this invention.

The invention also provides the use of a synthetic in vitro fed state gastric biorelevant dissolution medium for solubility and dissolution testing of drug products against a reference listed product and support in vivo BA/BE studies.

In addition to testing in vitro solubility and dissolution of drugs (New Chemical Entities and generic drugs), and dosage forms thereof in biorelevant media simulating fed state gastric fluids low-fat to explore the effect of food on drugs, the present invention provides in vitro fed state gastric media for compatibility and stability tests with, for example, probiotics, nutrients, vitamins, as well as gastric devices, including implants, stents and bands for weight control and reduction and dose dumping studies with ethanol.

### DETAILED DESCRIPTION OF THE INVENTION

The compositions of the invention are synthetic in vitro biorelevant concentrates, modelled after the amount of fat in meals with variable amounts of fat, and the resulting physicochemical properties of stomach fluids after consumption of the meal. Fats include triglycerides, diglycerides, monoglycerides, lecithin and/or lysolecithin. The term “biorelevant concentrate/composition” (also referred to as the “biorelevant precursor concentrate composition” herein) refers to the fact that the biorelevant concentrate composition does not itself necessarily mimic the physiological environment of the stomach. Rather, the readily water dispersible biorelevant concentrates are capable, upon dilution, dispersion or suspension in or with an aqueous medium, to prepare expediently reliable fed state gastric media with simple mixing (for example a magnetic stirrer) and without any high energy input. The resulting media comprise stable uniform fat dispersions readily filterable through a 0.45 micrometer filter. Primarily, the biorelevant media mimic the physiological and physicochemical functions of gastric fluids induced by consumption of the test meal, and for in vitro solubility and dissolution tests to ascertain food effects of drugs.

The substantially solid/solid-like concentrates typically contain between 1% and 10% by weight of water or aqueous medium.

The gel-like concentrates typically contain between 10% and 25% by weight of water or aqueous medium.

The fat dispersion/liquid concentrates typically contain between 25% and 70% by weight of water or aqueous medium.

The biorelevant precursor compositions of the invention are typically provided in a container. Typically, the container is a laminated pouch or sachet. This container can be (but is not limited to) a glass, suitable plastic bottle (HDPE, PE, PP, etc.), suitable metal bottle (aluminium, stainless steel). Typically, said sachet or pouch comprises from about 1 g to about 1500 g, for example from about 5 g to about 500 g, of said biorelevant concentrates. Containers up to, for example, 10 kg can also be used.

The biorelevant precursor composition of the invention can be provided in a kit together with inter alia compositions, for example solid dissolution compositions and/or concentrated buffer solution suitable, upon dispersion, dilution or suspension in an aqueous medium, for simulating, for example, simulated fed state gastric fluids of mammalian species (for example human, canine, rabbit, rodent, murine, simian, and porcine) at the desired physiological pH.

The kits may also contain filters to separate undissolved drug particles from the filtrate containing dissolved drug with pore diameters, for example between 0.2 to 1 micron and pre filters with pore diameters, for example between 1 to 10 micron selected from for example glass microfibre, PVDF, nylon or PES.

As described in more detail herein, the biorelevant concentrate compositions of the invention comprise uniformly dispersed fats comprising triglycerides and/or diglycerides and/or monoglycerides as well as mixtures of lecithin (diacyl phospholipids) and/or lysolecithin (monoacyl phospholipids) from diacylation, further comprising carbohydrates and/or sugar alcohols in the aqueous medium wherein the water content in said concentrate composition is between 1.0% and 70.0% by weight.

The compositions of the invention may also contain smaller amounts of bile salt components (<3.0%) to reflect the result of duodenal reflux.

The biorelevant precursor compositions are surprisingly stable and reproducible for preparing biorelevant fed state in vitro gastric media (FEDGAS). The unexpected and surprisingly robust physicochemical properties of the biorelevant precursor concentrates in on-going stability tests at 22° C. in excess of 9 months and at least 9 months at 40° C., point the way to making consistent in vitro fed state gastric media for reproducible dissolution testing of drugs and (other) industrial applications (See Case Study 2).

The media's predictive and user friendly properties rest chiefly with constant physicochemical parameters, for example particle size, fat components, buffer capacity, surface tension, osmolarity, wherein the weight ratio of weight of total fat and total carbohydrate contents in the medium is 20:1 to 1:20; alternatively or preferably 15:1 to 1:15, or 10:1 to 1:10, or 5:1 to 1:5, or 2:1 to 1:2.

The surface tension of the in vitro fed state gastric dissolution media is typically between 30 and 50 mN/m.

The in vitro fed state gastric media is readily filterable, with substantially uniform sub-micron particles consistently below 1000 nm, preferably below 500 nm, more preferably below 250 nm, still more preferably below 200 nm and typically below 175 nm, for example 150 nm, with narrow size distribution, and polydispersity index (pdi) consistently below 0.2. The consistent physicochemical properties (e.g. particle size, narrow distribution, surface tension, pH compatibility across the physiological pH of fed state gastric fluid between pH 1.5 and pH 7.5 and temperature stability around 37° C. for at least 6 hours) are important

Characteristically, the dissolution media can be readily filtered using 0.22 to 10 μm pore size filters, preferably 0.45 to 1.0 μm. At least 20 mL of the dissolution medium can be readily filtered manually using a 0.45 μm pore size GE Healthcare Whatman™ GD/X Glass Micro Fiber (GMF) Syringe Filters. Typically, the Z-average particle size using photon correlation spectroscopy) is below 200 nm and typically 175 nm. The size distribution reflected by polydispersity index is consistently below 0.2.

The biorelevant precursor composition of this invention is a substantially solid/solid-like concentrate, a viscous gel-like concentrate, or liquid fat dispersion concentrate, comprising at least one primary component selected from each of the following groups of primary components:

i) Triglyceride and/or diglyceride and/or monoglyceride or any combinations thereof (between 1-70% by weight, preferably 3-70% by weight, more preferably 5-70% by weight);

ii) Lecithin and/or lysolecithin (between 1-45% by weight, preferably 1-30% by weight, more preferably 1-15% by weight);

iii) Carbohydrate (between 15-70% by weight, preferably 20-60% by weight); and

iv) Water or other aqueous medium (between 1-70% by weight, preferably 1 to 66% by weight, preferably 1 to 60% by weight);

wherein the weight ratio of total fats (one or more primary components from each of groups i) and ii) combined):total carbohydrates (one or more primary components from group iii) combined) is between 20:1 to 1:20, alternatively or preferably 15:1 to 1:15, or 10:1 to 1:10, or 5:1 to 1:5, or 2:1 to 1:2;

and the weight ratio of glyceride:lecithin and/or lysolecithin is between 45:1 and 1:45, alternatively or preferably 30:1 and 1:30, or 15:1 to 1:15, or 10:1 to 1:10, or 8:1 to 1:8, or 7:1 to 1:7, or 7:1 to 1:3;

and in addition at least one additional component selected from at least one member of the group comprising or consisting of:
(i) fatty acids (between 0.01-15% by weight, preferably 0.1-10% by weight);
(ii) bile acid/salt (between 0.01-3% by weight, preferably 0.1-1% by weight);
(iii) enzymes (between 0.01-2% by weight, preferably 0.1-1.5% by weight);
(iv) cholesterol, sterols (between 0.01-5% by weight, preferably 0.01 to 2.5% by weight);
(v) buffer agents (between 0.01-4% by weight, preferably 0.1 to 2% by weight);
(vi) osmotic agents (between 0.01-10% by weight, preferably 1 to 8% by weight);
(vii) proteins (collagen, protein hydrolysates, amino acids) (between 0.01-30% by weight, preferably 0.1% to 25% by weight);
(viii) mucin (between 0.1-5% by weight, preferably −2.5% by weight);
(ix) viscosity modifier (between 0.1-5% by weight, preferably 0.1 to 2.5% by weight); and
(x) preservatives, stabilizers (between 0.01-3% by weight, preferably 0.1 to 1.5% by weight), such as anti-oxidants, chelating agents, buffers (inorganic or organic), and antimicrobials.

Unless indicated to the contrary, all percentages by weight (referred to above and below) are by dry weight.

### Glycerides

The biorelevant precursor compositions of the invention may comprise at least one triglyceride in an amount of from 1%-70% by weight, preferably 3%-70% by weight, preferably 5%-70% by weight. Any synthetic, semi-synthetic or natural triglyceride can be used, from any vegetable or animal source/origin. The triglyceride(s) can be liquid or solid at relevant temperatures, e.g. from 15° C. to 30° C., for example about 20° C. The triglyceride(s) can, for example, be selected from avocado oil, canola oil, coconut oil, corn oil, cottonseed oil, olive oil, palm oil, peanut oil, rapeseed oil, safflower oil, sesame oil, soya oil (soybean oil), and sunflower oil. Preferable triglycerides include oils which are liquid at 20° C., such as soya oil (soybean oil), olive oil and rapeseed oil, and oils which are solid at 20° C., such as coconut oil and palm oil. Most preferably, the triglyceride comprises olive oil, avocado oil, palm oil. The triglyceride may be preferably a single oil from the same source or combining oils from different sources/origins. Natural or semi-synthetic or synthetic medium chain triglycerides (MCT) containing fatty acids with between six and 12 carbons are within the definition of triglycerides in this invention.

In preferred embodiments, the fatty acid profile of the biorelevant dissolution medium comprises at least 60% C18 but can be matched to the fatty acid profile of different type of meals. The biorelevant precursor compositions of the invention may comprise products of partial lipolysis of at least one triglyceride component as defined herein.

The biorelevant precursor compositions may further comprise at least one diglyceride. Any suitable diglyceride may be used in an amount of from 1%-70% by weight, preferably 3%-70% by weight, preferably 5%-70% by weight. Any diglyceride which is a product of lipolysis of any triglyceride defined herein may be used. Typically, the diglyceride when used is glyceryl di oleate.

The biorelevant precursor compositions of the invention may also comprise at least one monoglyceride in an amount of from 1%-70% by weight, preferably 3%-70% by weight, preferably 5%-70% by weight of the total glycerides. Further, the amount of monoglyceride when included would typically not be more than 50% of the total glycerides. Any suitable monoglyceride may be used; particularly any monoglyceride which is a product of lipolysis of any triglyceride or diglyceride as defined herein may be used. Typically, the mono glyceride is glyceryl mono oleate.

The biorelevant precursor compositions of the invention may also comprise at least one fatty acid at no more than 15% by weight. Any suitable fatty acid may be used; particularly any fatty acid which is a product of lipolysis of any triglyceride, diglyceride or monoglyceride as defined herein may be used. Typically, the fatty acid is oleic acid.

### Lecithin and Lysolecithin

It is to be understood that the description of lecithin embraces phospholipid which is the main component group of lecithin, along with neutral lipids, for example, glycolipids, fatty acid, triglycerides amongst others. Phospholipids comprise chiefly phosphatidylcholine (PC). The purity of phospholipids/lecithin is conventionally linked to the amount of PC in the mixture; which mixture may also comprise phosphatides, such as phosphatidyl inositol, phosphatidyl serine, as examples.

Phospholipids (lecithin) can possess twin hydrocarbon tails and be identified as diacyl phospholipid. The molecule can also have only one hydrocarbon chain and be identified as mono acyl phospholipid. Monoacyl phospholipids are commonly known as lysolecithin. The hydrocarbon chain of the lecithin and lysolecithin can be saturated for example dimyristoylphosphatidylcholine and dimyristoylphosphatidylglycerol and/or unsaturated for example dioleoylphosphatidylcholine. The lecithin and lysolecithin further includes hydrogenated lecithin and lysolecithin for example hydrogenated soya lecithin. The lecithin and lysolecithin may be obtained synthetically, semi synthetically or from any vegetable or animal source, including but not limited to soy, egg, canola, rapeseed, sunflower or fish.

The biorelevant precursor concentrate compositions of the invention contain one or more phospholipid and/or one or more lysophospholipid as described. Any suitable lecithin (phospholipid) and/or lysophospholipid (lysolecithin) may be used from natural, semi-synthetic or synthetic sources. Charged phospholipids can improve stability of dispersed fat aggregates. Phospholipid (lecithin) comprises chiefly phosphatidylcholine (PC) along with smaller amounts of phosphatidylethanolamine (PE), phosphatidylserine (PS), phosphatidic acid (PA), phosphatidylinositol (PI), phosphatidylglycerol (PG). Lysophospholipid (lysolecithin) comprises chiefly lysophoshatidylcholine along with smaller amounts of the monoacyl derivative of the other phosphatides.

Biorelevant precursor concentrate compositions have phospholipid content comprising lecithin and/or lysolecithin between 1-45% by weight, preferably 1-30% by weight, preferably 1-15% by weight.

Of this mixture, the PC may be broadly from 15% to 99% by weight. The LPC may be between 0.5% to 85.0% by weight. Phospholipids comprising 30.0% to 95.0% PC and between 2.0% and 70.0% LPC are preferred. In this invention, the terms lecithin and phospholipid are interchangeable and include:
(i) both lecithin and lysolecithin or phospholipid and lysophospholipid in the mixture
(ii) either lecithin (phospholipid) or lysolecithin (lysophospholipid) by themselves.
   The amount of lecithin and lysolecithin together is between 30% and 98% by weight.

### Carbohydrate

The biorelevant precursor concentrate compositions of the invention comprise at least one carbohydrate and/or sugar alcohol. Any suitable carbohydrate such as a saccharide (commonly known as a sugar) by itself, and/or a suitable sugar alcohol can also be used. For example, the saccharide/sugar may be selected from a list of monosaccharides, for example fructose, glucose, galactose, mannose, ribose and the like; a list of disaccharides, for example sucrose, lactose, maltose, trehalose and the like; or combinations of mono and disaccharides.

The sugar alcohol may be selected from mannitol, lactitol, sorbitol and xylitol, and the like. The term sugar alcohol herein includes polyols such as glycerol.

Preferred concentrates comprise sugar by itself, and/or sugar alcohol selected from glucose, fructose, sucrose, lactose, erythritol, maltitol, isomaltol, mannitol or xylitol. Combinations of sugar(s) and/or sugar alcohol(s) can also be used.

Primary component group of carbohydrate components comprising saccharide (sugar), sugar alcohol, and combinations thereof in suitable proportions may be combinations of, for example, glucose (monosaccharide), fructose (sugar alcohol), sucrose (disaccharide), dextrin or starch (polysaccharides) providing the biorelevant precursor concentrate of this invention with a water activity below 0.86, preferably below 0.70, thereby inhibiting microbial growth and providing excellent long term storage of the precursor concentrates with a CFU below 10 (Table 2). For the solid/solid like concentrates the water activity is also below 0.7. Water activity is a parameter for industrial applicability and benefits offered by this invention in the field of biorelevant in vitro dissolution testing and simulation of fed state gastric fluids.

TABLE 2
Physicochemical properties of the biorelevant gel-like precursor concentrate.

| | |
|---|---|
| Microbiology (CFU/g) | <10 |
| Water activity (aw) | 0.5-0.8 |
| Average particle size (nm) | <200 |

If the water activity is greater than 0.86 or the CFU count is above for example >10 CFU/g, the microbial burden of the precursor concentrate composition may be reduced by, for example but not limited to, pasteurization, UHT, aseptic filtration, steam sterilization.

The biorelevant precursor concentrates of the invention may further comprise a viscosity modifier, including but not limited to an oligosaccharide and/or polysaccharide which may be digestible or non-digestible. For example, the polysaccharide can be starch, modified starch, dextrin, celluloses, polydextrose, pectin, galactomannans, alginates and the like, and/or semi synthetic versions such as methylcellulose, carboxymethylcellulose, hydroxypropyl methylcellulose, chitosan, and the like.

The precursor concentrates of the invention contain total fat:carbohydrate ratios within the range 20:1 to 1:20, preferably 15:1 to 1:15, preferably 10:1 to 1:10, preferably 5:1 to 1:5, preferably 2:1 to 1:2.

When the readily water dispersible precursor concentrates are diluted or dispersed in aqueous medium, the ratios in the resultant in vitro biorelevant fed state gastric dissolution media are maintained.

### Fatty Acids

The biorelevant precursor concentrates of the invention may comprise additional components further comprising free fatty acid, for example oleic acid, lauric acid, linoleic acid, stearic acid and palmitic acid and their salts.

### Bile Salts

The biorelevant precursor concentrates of the invention may comprise additional components, for example, a bile salt. Any suitable bile salt can be used. Suitable bile salts include sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids, and combinations thereof. Preferably, the bile salt is selected from sodium cholate, sodium taurocholate, and sodium glycocholate. More preferably, the bile salt is sodium taurocholate.

### Buffers

The biorelevant precursor concentrate of the invention may comprise buffer agents and osmotic agents. However, the buffer agents, preferably buffer concentrates or solutions, are incorporated/added to the biorelevant concentrate composition or the in vitro biorelevant fed state simulated dissolution media. More preferably, the buffer agents are added using dilutable concentrates that require from ×3 to ×60, preferably from ×5 to ×40, more preferably from ×15 to ×30 dilution.

The buffer concentrates may be incorporated into the biorelevant concentrate composition; or alternatively the said biorelevant concentrate composition may be incorporated into the buffer concentrate in reverse order to provide in vitro fed state dissolution medium at the required pH (pH 1.5 to pH 7.5), buffer capacity (5 to 100, preferably between 10 and 30, between 15 and 30 mM/ΔpH).

Further, purified water may be added to the mixture containing the biorelevant concentrate and dilutable buffer concentrate to prepare in vitro fed state dissolution media of the invention at the required target pH between pH 1.5 to pH 7.5 for dissolution testing. The biorelevant precursor concentrate composition, (ii) buffer concentrate and (iii) purified water can be added/combined in any order for preparing the in vitro fed state gastric dissolution media at the required pH for in vitro dissolution testing.

### Buffer Concentrate

A dilutable ×25 buffer concentrate containing the appropriate amounts of sodium chloride, citric acid and sodium citrate (amounts from Table 6) was prepared by dissolving the buffers and osmotic agent (sodium chloride) in purified water.

900 mL of the in vitro test media was prepared by
1) Weighing 36.8 g of ×25 buffer concentrate (pH3) into a suitable container
2) Adding 732.6 g of purified water

### 3) Adding 153.0 g of FEDGAS gel

4) Stirring until the dispersion is thoroughly homogeneous
The resulting in vitro test media has a pH of 3.0 and a buffer capacity of typically 22 mM/ΔpH.

The buffer concentrates and the biorelevant precursor concentrate compositions are in separate containers and combined in the manner described for preparing the in vitro fed state gastric media.

The two separate containers may be included in a kit along with, inter alia, filters for carrying out in vitro solubility and dissolution testing.

Any suitable buffer agent can be used. Suitable buffer agents include at least one inorganic buffer agent selected from monobasic sodium phosphate; acetic acid; hydrochloric acid; maleic acid; citric acid; lactic acid; potassium phosphate monobasic; trisodium citrate; sodium acetate trihydrate; imidazole; sodium carbonate; sodium hydrogen carbonate; sodium cacodylate; sodium barbital; phosphate salts such as Na₂HPO₄, NaH₂PO₄, K₂HPO₄ and KH₂PO₄; and sodium hydroxide, and/or at least one organic buffer agent selected from 2-(N-morpholino)ethanesulfonic acid (MES); Bis-tris methane (Bis Tris); 2-[(2-amino-2-oxoethyl)-(carboxymethyl)amino]acetic acid (ADA); N-(2-Acetamido)-2-aminoethanesulfonic acid (ACES); Bis-tris propane 1,3-bis(tris(hydroxymethyl)methylamino)propane; piperazine-N,N′-bis(2-ethanesulfonic acid) (PIPES); 2-(carbamoylmethylamino)ethanesulfonic acid (ACES); 2-Hydroxy-3-morpholinopropanesulfonic acid (MOPSO); Cholamine chloride; Cholamine chloride hydrochloride; 3-Morpholinopropane-1-sulfonic acid (MOPS); N N-bis 2-hydroxyethyl-2-aminoethanesulfonic acid (BES); 2-[[1,3-dihydroxy-2-(hydroxymethyl)propan-2-yl]amino]ethanesulfonic acid (TES); 2-[4-(2-hydroxyethyl)piperazin-1-yl]ethanesulfonic acid (HEPES); [3-Bis(2-hydroxyethyl) amino-2-hydroxypropane-1-sulfonic acid] (DIPSO); [3-Bis(2-hydroxyethyl) amino-2-hydroxypropane-1-sulfonic acid] MOBS; acetamidoglycine; 3-[[1,3-dihydroxy-2-(hydroxymethyl)propan-2-yl] amino]-2-hydroxypropane-1-sulfonic acid (TAPSO); 2,2′,2″-Nitrilotri(ethan-1-ol) (TEA); Piperazine-N,N′-bis(2-hydroxypropanesulfonic acid) (POPSO); 4-(2-Hydroxyethyl)piperazine-1-(2-hydroxypropanesulfonic acid) (HEPPSO); 4-(2-Hydroxyethyl)-1-piperazinepropanesulfonic acid (HEPPS); N-[Tris(hydroxymethyl)methyl]glycine (Tricine); tris(hydroxymethyl)aminomethane (Tris); glycinamide; glycine; glycylglycine; histidine; N-(2-Hydroxyethyl)piperazine-N′-(4-butanesulfonic acid) (HEPB S); 2-(Bis(2-hydroxyethyl)amino)acetic acid (Bicine); [tris(hydroxymethyl)methylamino]propanesulfonic acid (TAPS); 2-Amino-2-Methyl-1-Propanol (AMPB); 2-(Cyclohexylamino)ethanesulfonic acid (CHES); β-Aminoisobutyl alcohol (AMP); N-(1,1-Dimethyl-2-hydroxyethyl)-3-amino-2-hydroxypropane sulfonic acid (AMP SO); 3-(Cyclohexylamino)-2-hydroxy-1-propanesulfonic acid, CAPSO Free Acid (CAPSO); 3-(Cyclohexylamino)-1-propanesulfonic acid (CAPS); and 4-(Cyclohexylamino)-1-butanesulfonic acid (CABS).

### Osmotic Agents

Osmotic agents are included to adjust the osmolarity of the dissolution media to simulate the osmolarity of the fed state gastric fluids after a meal for example an FDA meal. Osmotic agents include but are not limited to typically sodium chloride, potassium chloride, calcium chloride, magnesium chloride, hydrochloric acid and sodium hydroxide, including combinations thereof. Carbohydrates and buffers may also contribute to the overall osmolarity of the in vitro dissolution media. The osmotic agents may be incorporated in the biorelevant precursor concentrate composition or preferably in the buffer concentrate. The osmolarity range of the fed state dissolution media is between 200 to 800 mOsm/L, typically 300 to 600 mOsm/L. After a meal, the osmolarity of the high-fat meal gastric fluids in the stomach is usually higher than after a low-fat meal. Furthermore, the osmolarity can be affected by the food contents and residence time in the fed stomach. The osmolarity of the in vivo fed state gastric fluids that can be simulated in the in vitro fed state gastric media of this invention are typically between 400 and 550 mOsm/L.

### Enzymes

If desired, additional components, for example enzymes such as gastric lipase and/or pepsin may be added to the actual dissolution media rather than in the concentrates.

### Preservatives and Stabilizers

Examples of anti-oxidants include but are not limited to ascorbic acid, ascorbyl palmitate, vitamin E and esters, carotenoids, vitamin A. Chelating agents include but are not limited to dimercaprol, disodium EDTA, desferrioxamine, citrate. Buffers (inorganic or organic) are as listed in the buffer section. Antimicrobials include but are not limited to thiomersal, sodium azide, butylated hydroxytoluene, butylated hydroxyanisol, sorbic acid.

In one preferred biorelevant precursor concentrate composition:
i) the at least one triglyceride is selected from soybean oil, olive oil, rapeseed oil, coconut oil, avocado oil and palm oil;
ii) the at least one diglyceride is selected from glycerol dioleate, glycerol distearate, glycerol dilaurate and linoleic diglyceride;
iii) the at least one monoglyceride is selected from glycerol monooleate, glyceryl mono stearate, glycerol monolaurate and linoleic monoglyceride;
iv) the at least one component selected from lecithin and/or lysolecithins comprises at least 40% phospholipid and/or at least 40% by weight lysophospholipid
vi) the at least one sugar and/or sugar alcohol comprises glucose and fructose and/or sorbitol
vii) the at least one polysaccharide comprises a starch, modified starch and/or dextrin,
   wherein the total fat:total carbohydrate ratio is 20:1 to 1:20, preferably 15:1 to 1:15, preferably 10:1 to 1:10, preferably 5:1 to 1:5, preferably 2:1 to 1:2; and, furthermore, the ratio of glyceride:lecithin and/or lysolecithin is between 45:1 and 1:45, preferably 30:1 and 1:30, preferably 15:1 to 1:15, preferably 10:1 to 1:10, preferably 8:1 to 1:8, preferably 7:1 to 1:7, preferably 7:1 to 1:3.

In particularly preferred biorelevant precursor compositions:
i) the at least one triglyceride comprises olive oil;
ii) the at least one diglyceride comprises glycerol dioleate;
iii) the at least one monoglyceride comprises glycerol monooleate;
iv) the at least one component selected from lecithin and/or lysolecithin comprises at least 15% by weight phosphatidylcholine (PC) and/or at least 0.5% lysophosphatidylcholine (LPC);
vi) the at least one sugar comprises glucose and fructose and sucrose;
vii) the at least one polysaccharide comprises a dextrin,

wherein the total amounts of fat:carbohydrate is 20:1 to 1:20, preferably 15:1 to 1:15, preferably 10:1 to 1:10, preferably 5:1 to 1:5 preferably 2:1 to 1:2; and, furthermore, the ratio of glyceride:lecithin and/or lysolecithin is between 45:1 and 1:45, preferably 30:1 and 1:30, preferably 15:1 to 1:15, preferably 10:1 to 1:10, preferably 8:1 to 1:8, preferably 7:1 to 1:7, preferably 7:1 to 1:3.

Variations in the biorelevant precursor composition of the invention can be made to meet desired physicochemical and in particular fat content of compositions which may be variants of for example the standard FDA high-fat meal.

It is within the scope of this invention to adjust and select total fat and carbohydrate contents in the concentrates for preparing dissolution media simulating fed state gastric media modelled-after meals with target fat values.

Typically, the water content of the composition is controlled between 1.0% and 25.0% to form either substantially solid/solid-like and gel-like liquid concentrates. Water removal by evaporation may be for example vacuum assisted or by freeze drying, for example lyophilisation.

The content of bile salt(s) in the composition of the invention is based on the amount of intestinal fluid regurgitated from the duodenum. When present, the amount of bile salts in the composition of the invention is below 3.0%, typically below 1.0% by weight.

By way of example and not by way of limitation, the following are typical examples illustrating the invention. A typical example of a concentrate composition is shown in Table 3 along with the range of typical components. The compositions may comprise lecithin and/or lysolecithins with different PC content (purity) in the mixtures.

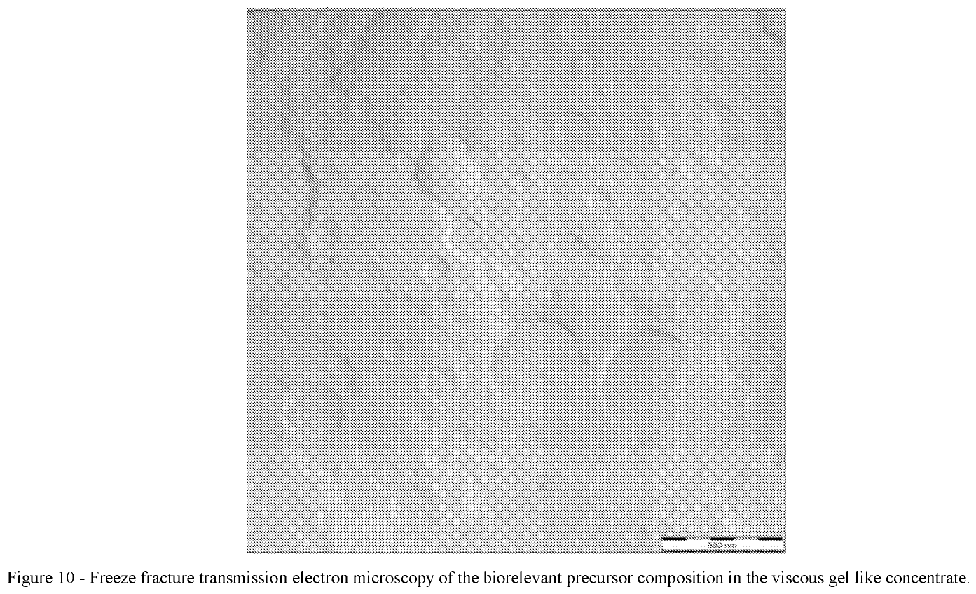

Freeze fracture of the biorelevant concentrate viscous gel-like precursor is shown in FIG. 10.

An example of the actual dissolution medium is shown in Table 8.

TABLE 3
Typical Composition of Precursor Composition/Concentrate

| Component | Concentration % (wt/wt) |
|---|---|
| Glycerides | 35 (between 1.0% and 70%) |
| Lecithin* | 4 (between 1.0% and 45%) |
| Carbohydrates | 39 (between 15.0% and 70%) |
| Bile salts | 0.2 (between 0.01% and 3%) |
| Stabilizers** | 0.9 (between 0.01% and 3%) |
| Water | 17.0 (between 1.0% and 70%) |

*(Lecithin and/or lysolecithin)
**Optional

Preparation and composition of in vitro dissolution medium prepared from the biorelevant concentrate composition shown in Table 3.

The amount of total fat in the biorelevant precursor concentrate of the invention is typically such that the fat concentration between 0.5% and 20% w/v is obtained when the precursor is dispersed, diluted or suspended in an aqueous medium to give a biorelevant medium.

More typically, the amount of fat in the biorelevant precursor concentrate is such that a fat concentration of from 4.0% w/v to 20.0% w/v, preferably 5% to 15% w/v, preferably 6% to 10% w/v, is obtained when the precursor composition is dispersed, diluted or suspended in an aqueous medium to give a biorelevant medium modelled on variants of FDA recommended standard meal with high-fat and low-fat amounts. Furthermore the amount of fat and amount used may be adjusted and selected in order to take into account other variants, namely medium-fat, low-fat variations, extremely high-fat contents up to 15% and extremely low-fat contents down to 0.1% are not outside the scope of this invention.

Table 3 sets out the primary components in a typical concentrate composition according to this invention. The variable fat contents of the desired in vitro test media may be obtained by selecting the components and adjusting amounts from said Table 3 thereby providing concentrated compositions for dilution and preparation of the test media for in vitro dissolution, solubility and stability tests of drugs and drug products. The tests in the in vitro dissolution media also evaluate the potential food effects on the drug and drug products due to the fat content at the physiological pH of the stomach or from beverages/drinks containing fat, for example tea with full-fat or reduced-fat milk.

### Typical Manufacturing Method of Precursor Compositions

### Step 1

The following components are weighed into a suitable reactor or processing vessel:
Purified water 66 kg
**Stabilizers 0.8 kg

### **Optional

Suitable reactors include but are not limited to evaporator, thin film evaporator, microwave, optionally vacuum assisted. The solution is stirred between 50 to 10000 RPM, preferably between 50 to 2000 RPM, preferably between 50 to 500 RPM, and maintained between 15-80° C., preferably between 30-80° C., more preferably between 40-70° C.

### Step 2

When the solution from step 1 is homogeneous, the following components are added:

| | |
|---|---|
| ***Lecithin | 4.50 kg |
| Bile salts | 0.16 kg |

***Lecithin and/or lysolecithin.

The suspension from step 2 is stirred between 50 to 30000 RPM, preferably between 100 to 10000 RPM, preferably between 200 to 5000 RPM at temperatures between 15-80° C., preferably between 40-70° C. A light vacuum is maintained until all the components are fully hydrated.

### Step 3

When the suspension from step 2 is homogeneous, the following component is added:

| | |
|---|---|
| ****Glycerides | 28 kg |

****triglyceride.

### Step 4

The suspension from step 3 is stirred between 50 to 30000 RPM, preferably between 100 to 10000 RPM, preferably between 200 to 5000 RPM at temperatures between 15-80° C., preferably between 30-80° C., preferably between 40-70° C. A light vacuum is maintained until all the components are fully mixed. A homogeneous fat dispersion with particle size of about 0.5 to 5 microns is obtained.

### Step 5

The fat dispersion from step 4 may be further processed using homogenizer selected from high shear mixers, high pressure, microfluidizer, ultrasonic or any other appropriate high energy homogenizer.
99.5 kg (yield) of the homogenised fat dispersion from step 5 and components shown in Table 4 is obtained with 1000 nm Z-average diameter, typically below about 500 nm.
The homogenised fat dispersion is transferred to a suitable holding tank or container.

TABLE 4
Typical Example of the components in
homogenized fat dispersion at the end of step 5.

| Components | Concentration % (w/w) |
|---|---|
| Water | 66 |
| Glycerides | 28 |
| Lecithin*** | 4.5 |
| Bile salts | 0.16 |
| Stabilizers** | 0.8 |

**Optional
***Lecithin and/or lysolecithin

### Step 6

The components below are added to the reactor in step 1:

| | |
|---|---|
| Carbohydrate*** | 22.10 kg |
| Dextrin | 1.16 kg |

***Invert sugar

The solution is stirred and heated between 20-80° C., typically between 50-70° C. A vacuum is applied to start evaporation, typically between 10 to 1000, preferably between 50 to 200 mbar. The water content at this stage is between 1 and 70%, typically between 15 and 30% by weight.

### Step 7

The homogenized fat dispersion, previously stored in the holding tank, is added continuously to the reactor. The fat dispersion is added at a controlled flow rate between 0.1 to 10 l/min, for example between 0.1 to 5 l/min, in keeping with the rate of evaporation under vacuum between 10 to 1000 mbar. During the continuous addition of the homogenized fat dispersion, the water content of the mixture inside the suitable reactor is between 5% and 70%, preferably maintained between 10 and 40%.
At the end of step 7, the concentrate composition is in the form of a substantially solid/solid-like concentrate, substantially gel-like concentrate or liquid fat dispersion/concentrate depending on the targeted water content generally between 1 and 70% by weight, between 10% and 25% for a viscous gel-like concentrate illustrated in Table 5, For a solid/solid-like concentrate the water content is typically between 1.0% and 10.0%, for a fat dispersion/liquid concentrate the water content is between 25% and 70%.

TABLE 5
Typical Example of precursor gel-like
composition at the end of step 7

| Components | Concentration % (w/w) |
|---|---|
| Water | 17 |
| Glycerides | 35.2 |
| Lecithin*** | 5.8 |
| Bile salts | 0.2 |
| Stabilizers** | 0.9 |
| Carbohydrates | 41.1 |

**Optional
***Lecithin and/or lysolecithin

### Packaging of the Precursor Composition

The gel-like concentrate/composition obtained in step 7 is filled into a suitable container. This container can be (but is not limited to) a sachet, a pouch, a suitable plastic bottle (HDPE, PE, PP, etc.), suitable metal bottle (aluminium, stainless steel, etc).
The composition is preferably packed under vacuum or sealed under an inert gas blanket, e.g. nitrogen.
The gel-like concentrate can be filled and/or packed in a single dose or a multi dose container, for example suitable containers of up to 10 kg capacity.

### Biorelevant Media Preparation

A synthetic aqueous biorelevant medium is obtained by adding an aqueous medium to the biorelevant gel-like composition/concentrate in Table 5 as described under the manufacturing method. The aqueous medium comprises, for example, but is not limited to, purified water, aqueous medium comprising buffers, osmotic components. Citrate buffers illustrated in the examples can be substituted by other combinations, for example acetic buffer for pH 5 and phosphate buffer for pH 3. Additional components, for example enzymes such as gastric lipase, may also be present, along with osmotic agents and buffers in the dissolution compositions of the invention.

Typically, the biorelevant dissolution medium is prepared as follows from the concentrate in Table 5 to make, for example, 900 ml of medium for a high-fat FDA meal:
1—Add approx. 600 g of water and suitable buffer components for the desired pH into a container.
2—Add 152.3 g of concentrate shown in Table 5 into the container.
3—Make up to volume by adding the rest of the purified water (763.62 g of total water).
4—Add magnetic stirrer and leave to stir until all the components have been thoroughly mixed.
5—Check pH 4.5.
   The following typical examples in Table 6, Table 7 and Table 8 can be obtained using the preparation method above.

TABLE 6
Typical example of a pH 3 biorelevant dissolution
medium produced from the biorelevant
concentrate and an aqueous buffer solution.

| | | | Individual | Component | Component |
|---|---|---|---|---|---|
| Component group | Component | Component | Component | group | group |
| | | (g) | % (w/w) | (g) | % (w/w) |
| Biorelevant | — | — | — | 152.30 | 13.56 |
| concentrate in | | | | | |
| Table 5 | | | | | |
| Buffer/salts | Sodium citrate | 0.54 | 0.06 | 7.16 | 0.62 |
| | dihydrate | | | | |
| | Citric acid | 4.45 | 0.48 | | |
| | Sodium chloride | 0.77 | 0.08 | | |
| Water (in buffer) | Water | 763.62 | 82.85 | 763.62 | 82.85 |
| TOTAL | | 921.67 | 100.00 | 921.67 | 100 |

TABLE 7
Typical example of a pH 4.5 biorelevant
dissolution medium produced from the biorelevant
concentrate and an aqueous buffer solution.

| | | | Individual | Component | Component |
|---|---|---|---|---|---|
| | | Component | Component % | group | group |
| Component group | Component | (g) | (w/w) | (g) | % (w/w) |
| Biorelevant | — | — | — | 152.30 | 16.50 |
| concentrate in | | | | | |
| Table 5 | | | | | |
| Table 5 - Typical | | | | | |
| Example of | | | | | |
| precursor gel-like | | | | | |
| composition at | | | | | |
| the end of step 7 | | | | | |
| Buffer/salts | Sodium citrate | 4.46 | 0.48 | 8.00 | 0.87 |
| | dihydrate | | | | |
| | Citric acid | 3.55 | 0.38 | | |
| Water (in buffer) | Water | 762.62 | 82.63 | 762.62 | 82.63 |
| TOTAL | | 922.92 | 100 | 922.92 | 100 |

TABLE 8
Typical example of a pH 6 biorelevant
dissolution medium produced from the biorelevant
concentrate and an aqueous buffer solution.

| | | | Individual | Component | Component |
|---|---|---|---|---|---|
| Component | | Component | Component | group | group |
| group | Component | (g) | % (w/w) | (g) | % (w/w) |
| Biorelevant | — | — | — | 152.30 | 16.53 |
| concentrate in | | | | | |
| Table 5 | | | | | |
| Buffer/salts | Sodium citrate | 6.74 | 0.73 | 7.68 | 0.83 |
| | dihydrate | | | | |
| | Citric acid | 0.943 | 0.10 | | |
| Water (in buffer) | Water | 787.51 | 85.45 | 787.51 | 82.64 |
| TOTAL | | 921.60 | 100.00 | 921.60 | 100.00 |

The dissolution medium can also be prepared from individual components by weighing them out separately into a buffer solution and then homogenising, as shown in Table 9a.

TABLE 9a
Typical dissolution medium comprising individual components in an
aqueous buffer solution at pH 4.5

| | Component | Component (g) | Component % (w/w) |
|---|---|---|---|
| Individual | Glycerides | 53.61 | 5.81 |
| components | Lecithin | 8.83 | 0.96 |
| | Bile salts | 0.30 | 0.03 |
| | Stabilizers** | 1.37 | 0.15 |
| | Carbohydrates | 62.60 | 6.78 |
| Buffer/salts | Sodium citrate | 4.46 | 0.48 |
| | dihydrate | | |
| | Citric acid | 3.55 | 0.38 |
| Water | Water | 788.20 | 85.40 |
| TOTAL | | 922.92 | 100 |

**Optional

The biorelevant dissolution medium shown in Table 9b is prepared as follows to make, for example, 900 ml of medium of a low-fat FDA meal:
1—Add approx. 600 g of water and suitable buffer components for the desired pH into a container.
2—Add 76.15 g of concentrate from Table 5 into the container.
3—Make up to volume by adding the rest of the purified water (889 g of water needs to be added in total).
4—Add magnetic stirrer and leave to stir until all the components have been thoroughly mixed.
5—Check pH 4.5.

TABLE 9b
Typical example of a pH 4.5 biorelevant dissolution medium simulating
a low-fat FDA meal produced from the biorelevant
concentrate and an aqueous buffer solution.

| | | | % (w/w) | | |
|---|---|---|---|---|---|
| | | | individual | Component | % (w/w) |
| | Component | Component (g) | Component | group (g) | Component group |
| Biorelevant | — | — | — | 76.15 | 8.25 |
| concentrate in | | | | | |
| Table 5 | | | | | |
| Buffer/salts | Sodium citrate | 4.46 | 0.48 | 8.00 | 0.87 |
| | dihydrate | | | | |
| | Sodium chloride | 3.55 | 0.38 | | |
| Water (in buffer) | Water | 838.77 | 90.88 | 838.77 | 90.88 |
| TOTAL | | 922.92 | 100.00 | 922.92 | 100 |

The biorelevant dissolution medium shown in Table 10 is prepared as follows to make, for example, 900 ml of medium of a double high-fat FDA meal:
1—Add approx. 500 g of water and suitable buffer components for the desired pH into a container.
2—Add 304.59 g of concentrate from Table 5 into the container.
3—Make up to volume by adding the rest of the purified water (610.33 g of water needs to be added in total).
4—Add magnetic stirrer and leave to stir until all the components have been thoroughly mixed.
5—Check pH 4.5.
   The following typical example in Table 10 is obtained using the preparation method above.

TABLE 10
Typical example of a pH 4.5 biorelevant dissolution medium simulating
a double high-fat FDA meal produced from the biorelevant
concentrate and an aqueous buffer solution.

| | | | % (w/w) | | % (w/w) |
|---|---|---|---|---|---|
| | | Component | individual | Component | Component |
| | Component | (g) | Component | group (g) | group |
| Biorelevant | — | — | — | 304.59 | 33.05 |
| concentrate in | | | | | |
| Table 5 | | | | | |
| Buffer/salts | Sodium citrate | 4.46 | 0.48 | 8.00 | 0.87 |
| | dihydrate | | | | |
| | Citric acid | 3.55 | 0.38 | | |
| Water (in buffer) | Water | 610.33 | 66.13 | 610.33 | 66.13 |
| TOTAL | | 922.92 | 100.00 | 922.92 | 100 |

Table 11 shows the typical physicochemical properties of the media prepared previously.

TABLE 11
Physicochemical properties of the
typical media prepared as shown for
preparing the dissolution media
shown in Table 9 and Table 10

| | | Average | | Surface | |
|---|---|---|---|---|---|
| | Buffer capacity | particle | Polydispersity | tension | Osmolarity |
| | (mmol/(l.ΔpH) | size (nm) | index | (mN/m) | (mOsm/L) |
| pH 3 | 22 | 160 | 0.14 | 40.6 | 435 |
| pH 5 | 27 | 160 | 0.11 | 41.3 | 451 |
| pH 6 | 30 | 160 | 0.14 | 39.3 | 479 |

### Biorelevant Dissolution Media for Simulating Fed State Gastric Fluid

The biorelevant dissolution medium can also be made from scratch by combining all the components in Table 5 with a predetermined amount of water to obtain the target content of the biorelevant dissolution medium. The fat content of this medium can vary from 20 to 100 grams in 500 ml of media (USP Dissolution Apparatus 2) depending on the amount of concentrate used in Table 5 and the dose of the drug in the dissolution test. Buffer salts and additional components can be added before or after the lipophilic components are dissolved or suspended in the aqueous medium.

However, dissolution media made from scratch do not have the stability and storage properties of concentrates. Thus, media prepared from scratch must be used within 24 hrs since they are prone to microbiological, physical and chemical spoilage, thereby making them less suitable and fit for purpose as dissolution media in terms of reliability, consistency and reproducibility.

### Case Studies

The case studies reported below demonstrate the usefulness of the present invention in evaluating food effects in the stomach on drug products.

In the case studies from 1 to 4, FEDGAS media at pH 6, pH 5 and pH 3 were prepared by adding the appropriate amount of purified water in a suitable container, adding the corresponding buffer concentrate and adding the appropriate amount of biorelevant precursor concentrate and mixing with a magnetic stirrer until homogeneous.

### Case Study 1—Dissolution of Exemestane (25 mg) Tablets (Brand Name: Aromasin, a Poorly Soluble Neutral Compound Immediate Release Formulation)

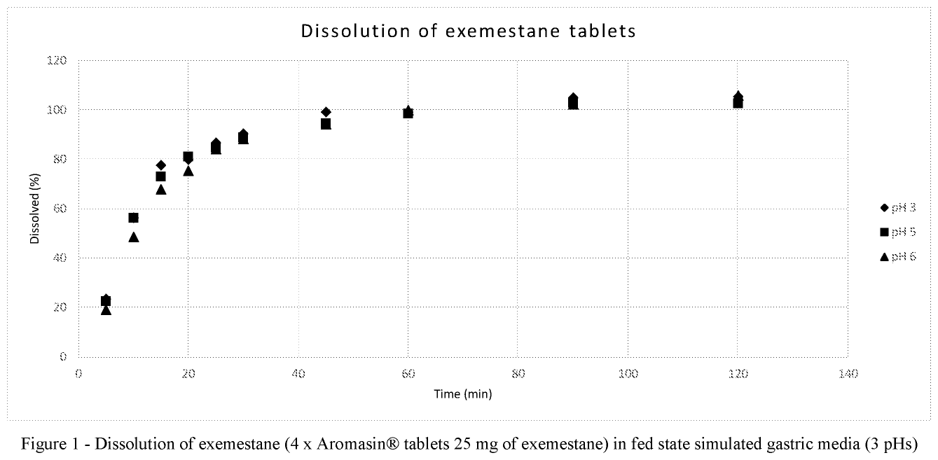

Three biorelevant fed state gastric media at pH 6, pH 5 and pH 3 using the composition in Table 5 were produced.
The media at these three pHs were characterised by measuring pH, buffer capacity, particle size (Z-average and polydispersity using Nanosizer) and surface tension (Kruss surface tension K6).
The media were stable at time zero and physically stable after 24 hours. Similarly, the key physicochemical properties were unchanged after 24 hours.
The dissolution of exemestane (4 tablet×25 mg tablets) in the media was carried out using USP 2 Dissolution Apparatus at 75 rpm (n=6 vessels). Samples of the three biorelevant media were taken from the dissolution vessels and filtered through a 0.45 micrometer nylon filter with prefilter at 5 mins, 10 mins, 15 mins, 20 mins, 25 mins, 30 mins, 45 mins, 60 mins, 90 mins and 120 mins and analysed for exemestane content by HPLC.
The results of these dissolution studies are provided in FIG. 1.
In line with the neutral chemical structure of exemestane, as can be seen exemestane was not sensitive to different pHs and the three dissolution profiles were very similar. Within 30 minutes more than 80% of the drug was dissolved across the three media.

### Case Study 2—Fed State Simulated Gastric Concentrate Stored for 9 Months at 40° C.

The biorelevant concentrate was stored at 22° C. and 40° C. for nine months and the study in Case Study 1 was repeated with media prepared from stored precursor concentrate. The ease of dispersibility in aqueous media of the fresh and stored precursor concentrate were very similar.
Unexpectedly, the dissolution profiles in the test media across the three pHs were found to be the same as when the media were prepared from freshly made precursor concentrate.

### Case Study 3—Dissolution of Cinnarizine (Basic Drug)

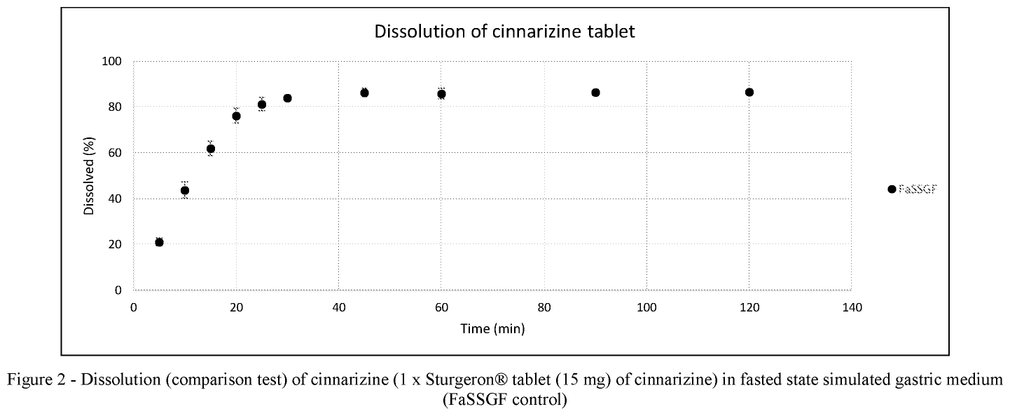

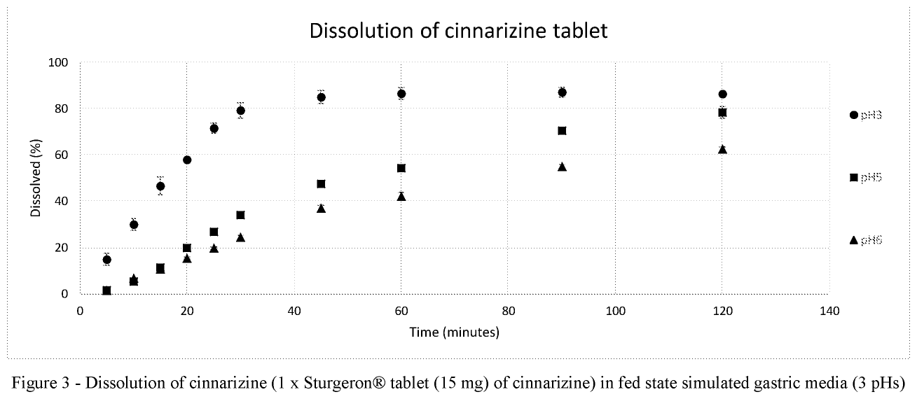

The dissolution of Sturgeron (15 mg cinnarizine immediate release tablets) was tested in a fasted gastric media (control experiment) and compared with fed gastric media at pH 6, pH 5 and pH 3 using the same dissolution set up as described previously.
The dissolution profiles in fasted state gastric media are provided in FIG. 2.
The dissolution profile of this drug product in the fasted gastric media indicates this basic drug cinnarizine dissolves rapidly in the fasted stomach. This control experiment shows that within 30 minutes close to 90% of the drug is dissolved.
In contrast, as seen in FIG. 3, this basic drug exhibits slower drug dissolution in all fed state gastric media (across the pH range from pH 6 down to pH 3). There is also a trend for the cinnarizine dissolution to be faster at lower pH (i.e. at a longer residence time of the drug in simulated fed state media, the dissolution rate increases). Reflecting the pH of the gastric fluids towards the end of gastric emptying.
These in vitro dissolution profiles can further be used with modelling software to better simulate how drugs behave in the stomach and how the drug (dissolved or suspended) is presented to the small intestine as the stomach empties. In combination with dissolution profiling in small intestinal fluids (fed and fasted), these inputs can lead to more accurate prediction in vitro in vivo correlations leading to more efficient development of a drug product.

### Case Study 4—Dissolution of Mefenamic Acid (Acidic Drug)

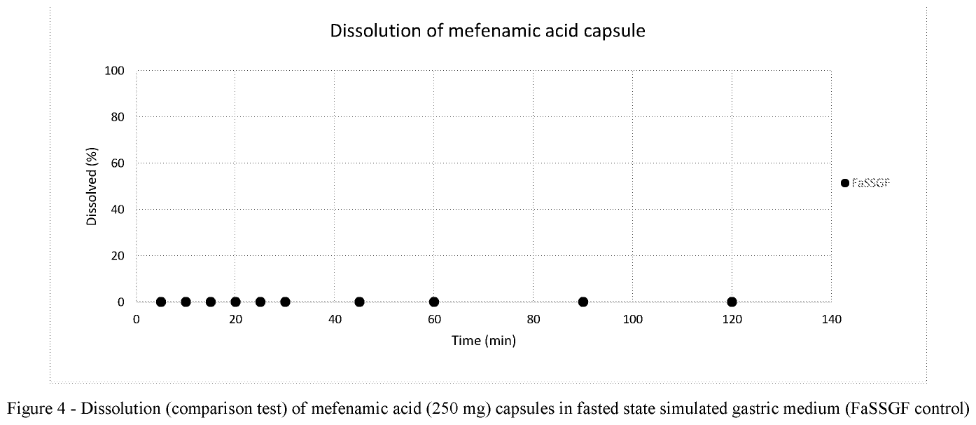

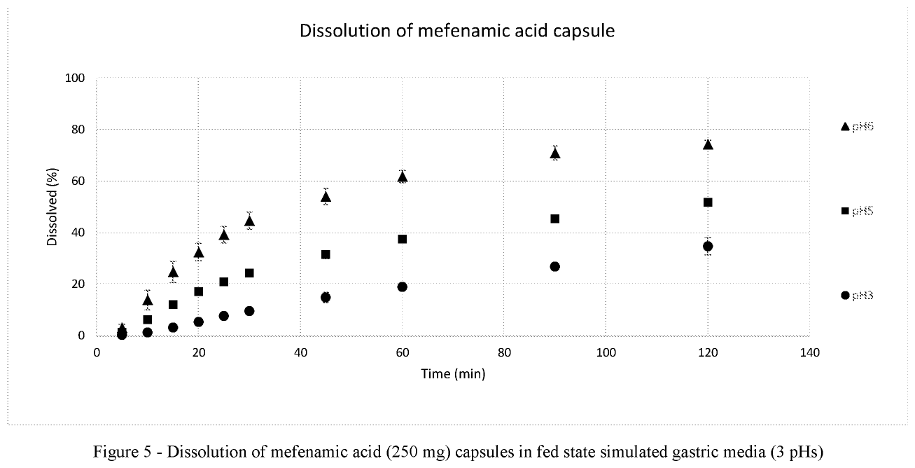

The dissolution of mefenamic acid hard gelatine capsules was carried out in fasted state gastric media (control) and compared with biorelevant fed state gastric media at pH 6, pH 5 and pH 3 as described using the set up and method in the Case Study 1.
Referring to FIG. 4, the dissolution in fasted state gastric media is negligible even after 2 hours of dissolution. Less than 0.1% of the drug dose is dissolved even after 2 hours of dissolution.
In sharp contrast to fasted state gastric media, in biorelevant fed state gastric media mefenamic acid dissolution proceeds considerably more rapidly (see FIG. 5). Dissolution reaches about 45%, 25% and 10% of the dosage form within 30 minutes in fed state gastric media at pH 6 (simulating fed conditions immediately after ingestion FDA high-fat meal), pH 5 (simulating fed conditions approximately 1 to 3 hours after ingestion of FDA high-fat meal) and pH 3 (simulating fed conditions approximately 4 to 5 hours after ingestion of FDA high-fat meal) respectively.

### Case Study 5—Dissolution of Danazol Capsules Acid (Neutral Drug)

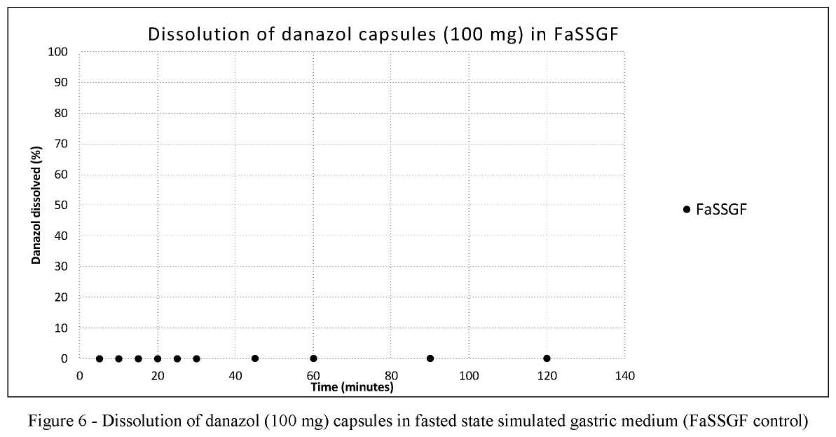

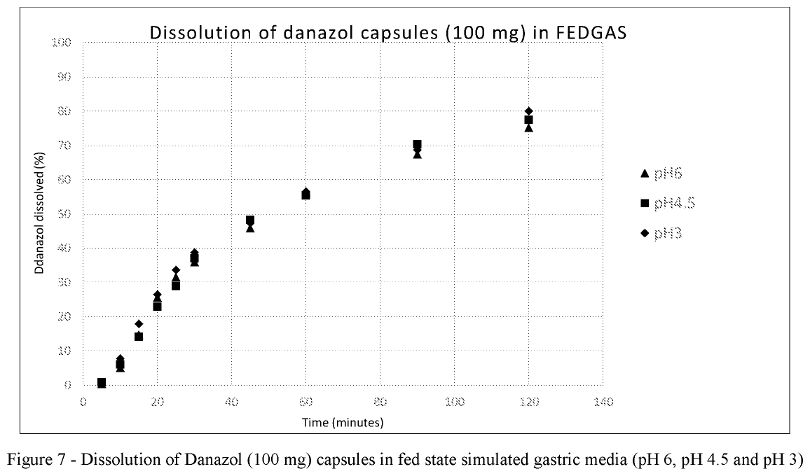

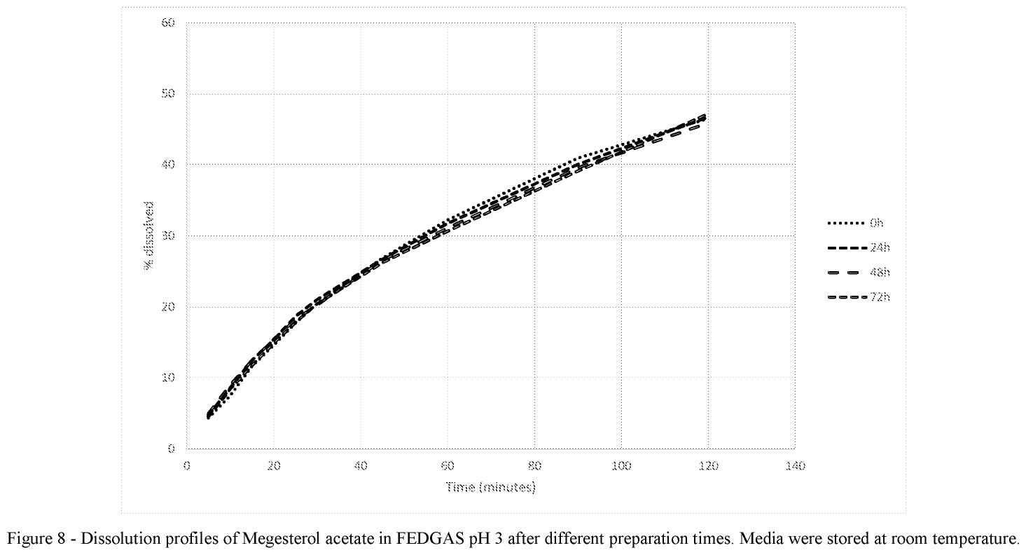

The dissolution of danazol (100 mg) hard gelatine capsules was carried out in fasted state gastric media (control) and compared with biorelevant fed state gastric media at pH 6, pH 4.5 and pH 3 as described using the set up and method in the Case Study 1.
Referring to FIG. 6, the dissolution in fasted gastric media is negligible even after 2 hours of dissolution. Less than 0.2% of the drug dose is dissolved even after 2 hours of dissolution.
In sharp contrast to fasted state gastric media, in the biorelevant fed state gastric media danazol dissolution proceeds considerably more rapidly (see FIG. 7). Dissolution reaches about 55% of the dosage form within 60 minutes in fed state gastric media at pH 6, pH4.5 and pH 3 respectively.
Case Study 6—Examining Physicochemical Properties of FEDGAS Dissolution Media Stored for 72 Hrs at Room Temperature with Dissolution of Megesterol Acetate Capsules
FEDGAS media at pH 6, pH 4.5 and pH 3 were prepared by adding the appropriate amount of water in a suitable container, adding the corresponding buffer concentrate and adding the appropriate amount of readily water dispersible biorelevant precursor concentrate and mixing with a magnetic stirrer until homogeneous. The three media were stored at room temperature for up to 72 hours. Dissolution in each of the media at t=0, t=24 hours, t=48 hours and t=72 hours after media preparation was carried out using megesterol acetate capsules (160 mg) in 900 mL of medium in the vessels. The results of the dissolution tests are provided in FIG. 8. The pH, buffer capacity, surface tension and particle size (Z-average) of the FEDGAS pH 6 and FEDGAS pH 3 were measured at t=0 and at t=72 hours. The results are provided in Table 12.
The results indicate that the dissolution profiles of megesterol acetate hard gelatin capsules for all three media at the four time points were identical. This study indicates that the 3 media did not age after three days of storage. The pH, buffer capacity, surface tension and particle size at t=72 hours were close to the values at t=0 hours.

TABLE 12
Biorelevant media properties after preparation according to the
examples. Media was stored for 72 h at room temperature.

| | | | | | Surface | | | |
|---|---|---|---|---|---|---|---|---|
| | | | Buffer capacity | | tension | | Particle size | |
| Biorelevant | pH | | (mmol/(ΔpH · L)) | | (mN/m) | | (nm) | |
| media | 0 h | 72 h | 0 h | 72 h | 0 h | 72 h | 0 h | 72 h |
| Early | 5.99 | 6.00 | 25.0 | 25.5 | 41 | 43 | 151.8 | 151.2 |
| FEDGAS | | | | | | | | |
| pH 6 | | | | | | | | |
| Late | 2.99 | 3.01 | 22.8 | 22.7 | 41 | 43 | 151.1 | 153.1 |
| FEDGAS | | | | | | | | |
| pH 3 | | | | | | | | |

The invention provides biorelevant dissolution compositions and methods of obtaining simulated media from precursor concentrates for in vitro dissolution and solubility studies. The simulated gastric media are modelled after stomach contents following consumption of high and low-fat meals and alternatives, containing even higher fat (up to 200 g of fat) and even lower fat amounts (1 g of fat). The invention fills a practical need for fed-state biorelevant testing alongside fasted state media, permitting more precise in vitro assessments after meal intake such as food effect on drugs after a FDA standard meal. Food effects also include in vitro dissolution testing of a drug product in simulated gastric fluids (FEDGAS) containing for example 1 g of fat that is in for example in a cup of milk tea supporting improved in vitro-in vivo correlations. Use of said biorelevant media rather than prior art media is compelling and advantageous; in particular, for the characterisation of pharmacologically active/relevant substances such as drug compounds, oral dosage forms, and the like. Furthermore, examining food effects in the stomach fills a practical need for characterisation of drugs, for example in lead optimisation as well as generic formulation development thereby leading to cost and time savings.

The invention provides unexpectedly stable, readily water dispersible biorelevant concentrate compositions. The biorelevant concentrate and buffer concentrate compositions can be used to produce a readily filterable and surprisingly stable biorelevant test media which simulate fed state stomach fluids after consumption of for example high-fat to low-fat FDA meals. The invention is thereby clearly advantageous and has industrial applicability. The proposed fed state simulated gastric fluids can be used in biorelevant dissolution and solubility studies for profiling drugs and drug products.

1. A biorelevant precursor composition suitable, upon dispersing, dilution or suspension in an aqueous medium, for simulating fed-state gastric fluids of mammalian species, wherein said biorelevant precursor composition comprises a substantially solid/solid-like concentrate, a viscous gel-like concentrate, or a liquid fat dispersion/concentrate, comprising at least one primary component selected from each of the following groups of primary components comprising:
i) Triglyceride and/or diglyceride and/or monoglyceride or any combinations thereof in an amount of from 1-70% by weight;
ii) Lecithin and/or lysolecithin in an amount of from 1-45% by weight;
iii) Carbohydrate in an amount of from 15-70% by weight; and
iv) Water or other aqueous medium in an amount of from 1-70% by weight;
wherein the weight ratio of total fats (one or more primary components from each of groups i) and ii) combined):total carbohydrates (one or more primary components from group iii) combined) is between 20:1 to 1:20; and
the weight ratio of glyceride:lecithin and/or lysolecithin is between 45:1 and 1:45; and
in addition at least one additional component selected from at least one of the following:
(i) fatty acids (between 0.01-15% by weight);
(ii) bile acid/salt (between 0.01-3% by weight);
(iii) enzymes (between 0.01-2% by weight);
(iv) cholesterol, sterols (between 0.01-5% by weight);
(v) buffer agents (between 0.01-4% by weight);
(vi) osmotic agents (between 0.01-10% by weight);
(vii) proteins (collagen, protein hydrolysates, amino acids) (between 0.01-30% by weight);
(viii) mucin (between 0.1-5% by weight);
(ix) viscosity modifier (between 0.1-5% by weight); and
(x) preservatives, stabilizers (between 0.01-3% by weight), such as
a) anti-oxidants,
b) chelating agents,
c) buffers (inorganic or organic), and
d) antimicrobials;
all percentages being by weight.

2. A composition according to claim 1, wherein the at least one triglyceride is selected from avocado oil, canola oil, coconut oil, corn oil, cottonseed oil, olive oil, palm oil, peanut oil, rapeseed oil, safflower oil, sesame oil, soya oil, sunflower oil, and combinations thereof.

3. A composition according to claim 1, comprising at least one phospholipid and/or at least one lysophospholipid.

4. A composition according to claim 1, wherein the at least one carbohydrate comprises polysaccharide, oligosaccharide, disaccharide, monosaccharide and/or sugar alcohol.

5. A composition according to claim 4, wherein the sugar is selected from glucose, fructose and sucrose, and the polysaccharide is selected from starch, dextrin and cellulose.

6. A composition according to claim 1, which is in the form of a solid or solid-like concentrate containing between 1% and 10% by weight of water or aqueous medium.

7. A composition according to claim 1, which is in the form of a viscous gel-like aqueous concentrate containing between 10% and 25% by weight of water or aqueous medium.

8. A composition according to claim 1, which is in the form of a liquid emulsion concentrate containing between 25% and 70% by weight of water or aqueous medium.

9. A composition according to claim 1, wherein the biorelevant precursor concentrate has a water activity below 0.86, preferably below 0.70.

10. A method of producing a biorelevant precursor composition as claimed in claim 1, which comprises processing
i) from 1-70% by weight of at least one triglyceride, and/or at least diglyceride and/or at least one monoglyceride;
ii) from 1-45% by weight of at least one lecithin and/or lyso-lecithin;
iii) from 15-70% by weight of at least one carbohydrate; and
iv) from 1-70% of water or other aqueous medium;
wherein the ratio of total fat (components i) and ii) combined):total carbohydrate is from 20:1 to 1:20, and the ratio of glyceride:lecithin and/or lyso-lecithin is from 45:1 to 1:45,
and in addition at least one additional component selected from at least one of the following:
(i) fatty acids (between 0.01-15% by weight);
(ii) bile acid/salt (between 0.01-3% by weight);
(iii) enzymes (between 0.01-2% by weight);
(iv) cholesterol, sterols (between 0.01-5% by weight);
(v) buffer agents (between 0.01-4% by weight);
(vi) osmotic agents (between 0.01-10% by weight);
(vii) proteins (collagen, protein hydrolysates, amino acids) (between 0.01-30% by weight);
(viii) mucin (between 0.1-5% by weight);
(ix) viscosity modifier (between 0.1-5% by weight); and
(x) preservatives, stabilizers (between 0.01-3% by weight), such as
a) anti-oxidants,
b) chelating agents,
c) buffers (inorganic or organic), and
d) antimicrobials;
by means of dispersing and/or homogenisation and/or control of water content by evaporation and/or addition or titration, to obtain a substantially solid/solid-like concentrate, a viscous gel-like concentrate, or a liquid fat dispersion/concentrate.

11. A method as claimed in claim 10, which comprises the use of controlled evaporation after addition of water containing at least one carbohydrate or directly controlling the addition of water containing at least one carbohydrate.

12. A method of producing a composition suitable for simulating fed-state gastric fluids of mammalian species, which method comprises dispersing, diluting or suspending in an aqueous medium a biorelevant precursor composition as claimed in claim 1.

13. A method as claimed in claim 12, which method comprises dispersing or diluting the biorelevant precursor concentrate composition in an aqueous medium or buffer concentrate that can be added/combined in any order for preparing the in vitro fed state gastric dissolution media at the required pH for in vitro dissolution testing.

14. A method as claimed in claim 12, wherein the aqueous medium for dispersing or diluting the biorelevant concentrate composition for preparing the in vitro fed state dissolution medium may comprise:
(i) a buffer solution to reach the required pH, buffer capacity and osmolarity of the biorelevant dissolution media without further dilution; or
(ii) a sufficient weighed/measured amount of the biorelevant concentrate, a sufficient weighed/measured amount of the buffer concentrate, and purified water to reach the required pH, buffer capacity and osmolarity, in any order of incorporation and/or dilution.

15. A method of preparing a fed state gastric media as claimed in claim 12, which further includes adding enzymes, protein hydrolysates and/or other additional components as a separate step.

16. A method as claimed in claim 12, wherein the resulting dissolution media can be readily filtered using 0.22 to 10 μm pore size filters, the Z-average particle size using photon correlation spectroscopy is below 200 nm and typically 175 nm, and the size distribution reflected by polydispersity index is consistently below 0.2.

17. A kit for use in producing a composition suitable for simulating fed-state gastric fluids of mammalian species, which kit comprises an aqueous medium or a buffer concentrate in a first container together with a biorelevant precursor concentrate composition in a second container as claimed in claim 1.

18. A method of making a synthetic biorelevant in vitro fed state dissolution medium based on the amount of fat in high fat to low fat meals comprising adding an aqueous medium, buffered to from pH 1.5 to pH 7.5, or adding from ×3 times to ×60 times buffer concentrate to the biorelevant precursor concentrate composition of claim 1.
